(19) United States
(12) Patent Application Publication
(10) Pub. No.: US 2022/0228092 A1
(43) Pub. Date: Jul. 21, 2022

(54) SINGLE-USE, FLEXIBLE BIOPROCESSING BAG AND METHOD OF MANUFACTURING A SINGLE-USE, FLEXIBLE BIOPROCESSING BAG

(71) Applicant: GLOBAL LIFE SCIENCES SOLUTIONS USA LLC, MARLBOROUGH, MA (US)

(72) Inventors: JONATHAN KENNEY, LAKEVILLE, MA (US); HANNA-LEENA SAUKKONEN, BOSTON, MA (US); MICHAEL MILLER, SHREWSBURY, MA (US)

(73) Assignee: GLOBAL LIFE SCIENCES SOLUTIONS USA LLC, MARLBOROUGH, MA (US)

(21) Appl. No.: 17/606,584

(22) PCT Filed: Apr. 29, 2020
(86) PCT No.: PCT/EP2020/061847

§ 371 Date: Oct. 26, 2021

Related U.S. Application Data
(60) Provisional application No. 62/841,867, filed on May 2, 2019.

(51) Int. Cl. C12M 1/00

(52) U.S. Cl. CPC C12M 23/14; C12M 23/28

(57) ABSTRACT

A bioprocessing bag includes a plurality of panels joined to one another about respective edges of the plurality of sheets, by side welds, and a top panel joined to upper edges of the plurality of panels by a flat/convergence weld.

### CROSS REFERENCE TO RELATED APPLICATIONS

This application is a national stage of International Application No. PCT/EP2020/061847 filed on Apr. 29, 2020, which claims priority to U.S. Provisional Patent Application No. 62/841,867 filed on May 2, 2019, all of which are hereby incorporated by reference in their entireties.

### BACKGROUND

### Technical Field

Embodiments of the invention relate generally to bioprocessing systems and methods and, more particularly, to a single-use, flexible bioprocessing bag for use in a bioreactor vessel and a method of manufacturing a single-use, flexible bioprocessing bag.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless-steel shell or vessel. Use of sterilized disposable bags eliminates time-consuming step of cleaning of the vessel and reduces the chance of contamination. The bag may be positioned within the rigid vessel and filled with the desired fluid for mixing. Depending on the fluid being processed, the system may include a number of fluid lines and different sensors, probes and ports coupled with the bag for monitoring, analytics, sampling, and fluid transfer. For example, a plurality of ports may typically be located at the front of the bag and accessible through an opening in the sidewall of the vessel, which provide connection points for sensors, probes and/or fluid sampling lines. In addition, a harvest port or drain line fitting is typically located at the bottom of the disposable bag and is configured for insertion through an opening in the bottom of the vessel, allowing for a harvest line to be connected to the bag for harvesting and draining of the bag after the bioprocess is complete.

Existing single-use, flexible bioprocessing bags may take a variety of shapes and configurations. In applications where such bags are used in generally cylindrical bioreactor vessels, the flexible bags may likewise be cylindrical. Such bags are typically manufactured by overlapping the edges of adjoining sheets of material and welding such sheets to one another to form the cylindrical sidewall of the bag. To close off the top of the bag, a tri-seam weld is often utilized, which brings together the upper edges of each sheet of material used in the bag construction. A depiction of a typical tri-seam weld 10 is illustrated in FIGS. 1 and 2 (illustrating two tri-seam welds). The tri-seam weld at the top of the bag, however, forms sharp angles, causing stress concentrations when the bag is under pressure. Accordingly, this area of the bag, due to the presence of the tri-seam weld can be a potential failure point for leaks.

In view of the above, there is a need for a single-use flexible bioprocessing bag that is constructed in a manner so as to minimize stress concentrations at joining locations between the sheets of material, and a method of manufacturing such a single-use, flexible bioprocessing bag.

### BRIEF DESCRIPTION

In an embodiment, a bioprocessing bag includes a plurality of panels joined to one another about respective edges of the plurality of sheets, by side welds, and a top panel joined to upper edges of the plurality of panels by a convergence weld.

### DRAWINGS

The present invention will be better understood from reading the following description of non-limiting embodiments, with reference to the attached drawings, wherein below:

FIG. 1 is a schematic illustration shown a prior art tri-seam weld for a flexible bioprocessing bag.

FIG. 2 is a perspective view showing a prior art tri-seam weld.

FIG. 3 is perspective illustration of a flexible bioprocessing bag according to an embodiment of the invention.

FIG. 4 is a top plan view of the flexible bioprocessing bag of FIG. 3.

FIG. 5 is a plan view of a panel of the flexible bioprocessing bag of FIG. 3.

FIG. 6 is a perspective view showing assembly of panels to form the flexible bioprocessing bag.

FIGS. 7 and 8 are schematic illustrations showing assembly of a top plate to the flexible bioprocessing bag.

FIG. 9 is a schematic illustration of a prior art bioprocessing bag constructed using a tri-seam weld.

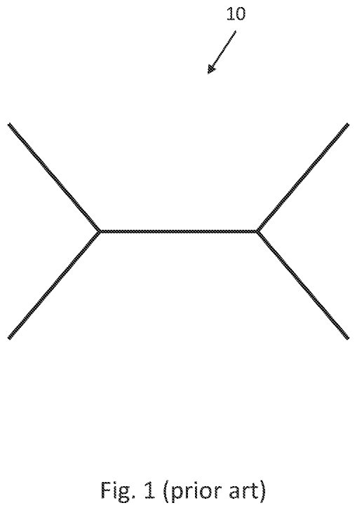

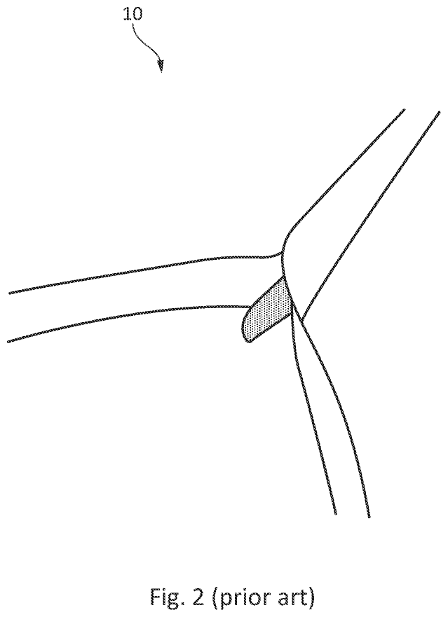

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within.

Embodiments of the invention provide single-use, flexible bioprocessing bags. In an embodiment, a bioprocessing bag includes a plurality of panels joined to one another about respective edges of the plurality of sheets, by side welds, and a top panel joined to upper edges of the plurality of panels by a flat weld, also called a convergence weld.

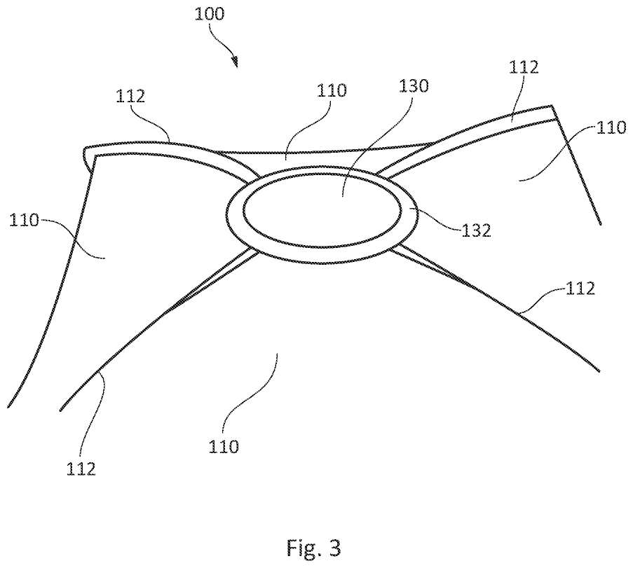

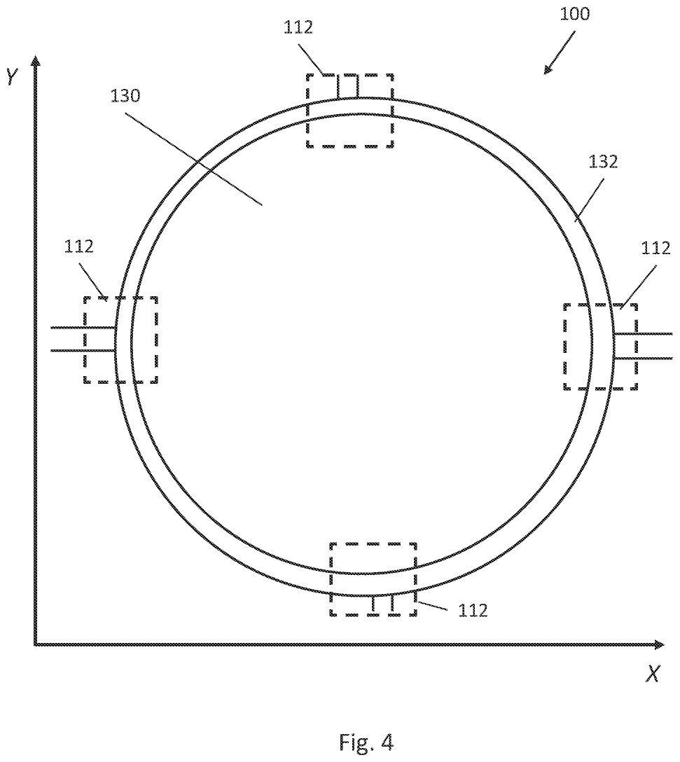

With reference to FIGS. 3 and 4, a flexible, single-use bioprocessing bag 100 according to an embodiment of the invention is illustrated. In an embodiment, the bag 100 is formed from four panels or sheets of material 110 that overlap and are joined about their respective edges by side seals/welds 112 (defining seams between the sheets of material), and a top plate or panel 130 that is joined to the upper edges of the sheets of material 110 by a flat/convergence weld 132. In an embodiment, a bottom panel (not shown) may be joined to the lower edges of the sheets of material in a similar manner. While FIGS. 3 and 4 illustrate the use of 4 sheets that form the sidewalls of the flexible bioprocessing bag 100, fewer than four sheets or more than four sheets may be utilized without departing from the broader aspects of the invention.

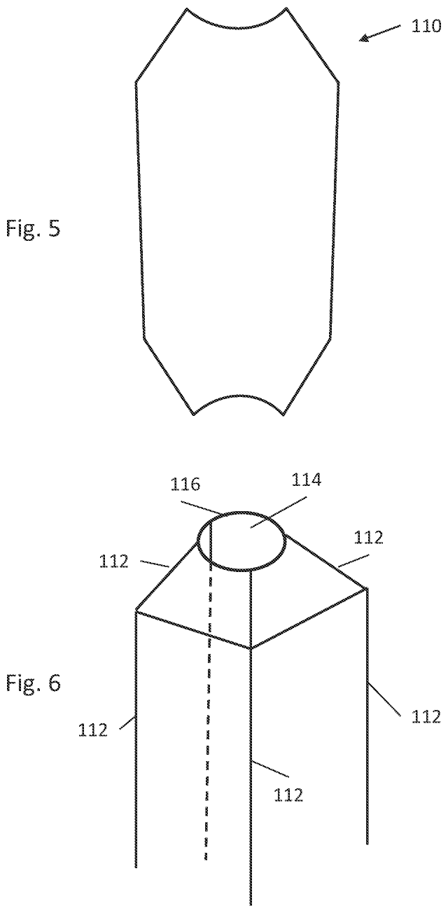

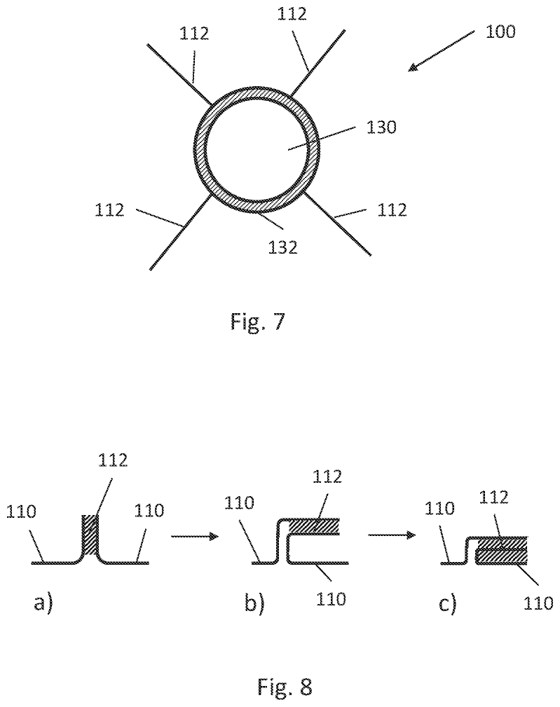

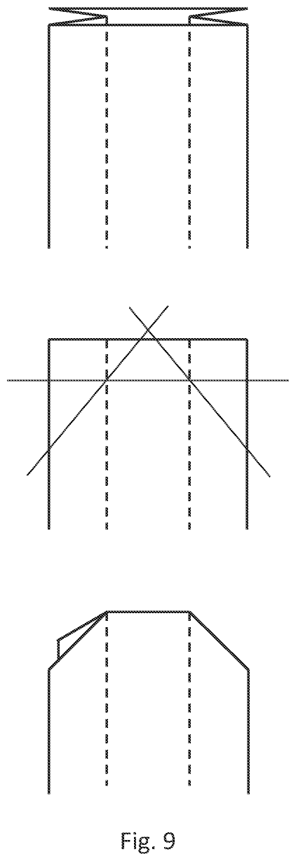

In an embodiment, the flexible bioprocessing bag 100 may be manufactured by first cutting each sheet of material 110 to the shape best illustrated in FIG. 5 (e.g., a generally rectangular shape with chamfered corners and optionally with circle segment cutouts at the short ends). The sheets 110 are then joined about their edges via the side welds 112 to define an interior space 114, as illustrated in FIG. 6. In an embodiment, the side welds 112 are continuous or substantially continuous welds that extends the length of the sheets 110. As best shown in FIG. 6 joining the sheets of material 110 to define the interior space forms a generally circular top opening 116. In some embodiments, depending on the specific configuration and shape of the sheets 110, the circular top opening 116 may be formed (e.g., by cutting) the sheets 110 after joining. With reference to FIGS. 7 and 8, the seams of the bag 100 (i.e., formed by joining the sheet 100 via the side welds) are then folded over (the middle drawing in FIG. 8), and the top plate 130 is placed atop the opening 116. The top plate 130 is then welded to the top edges of the sheets 110 (and folded-over seams) using a flat/convergence weld in the z-plane (as shown in FIG. 4). In an embodiment, the flat/convergence weld 132 is a continuous or substantially continuous annular weld that joins the top plate 130 to the sheets 110 to close off the top opening 116.

In this respect, the flat/convergence weld 132 occurs in a plane that is different than the rest of the weld (i.e., the side welds 112). This is in contrast to traditional bags which utilize a tri-seam weld, where all of the panels are welded together in a single direction (namely, the Y-plane). FIG. 9 illustrates a traditional tri-seam welding process. With the invention described herein, four single panels are welded in one plane (the Y plane according to the axis in FIG. 4), and then the top weld 132 is created in the Z-plane.

It is envisioned that the flat/convergence weld 132 may be accomplished by welding against a hard or rigid component such as an impeller base or manifold plate.

In embodiments, the single-use, flexible bag 100, and the sheets of material 110 thereof, may be formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low-density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™′ Bioclear™ 10 and Bioclear 11 laminates, available from GE Healthcare Life Sciences. Portions of the flexible container can comprise a substantially rigid material such as a rigid polymer, for example, high density polyethylene, metal, or glass. The flexible bag may be supplied pre-sterilized, such as using gamma irradiation.

In an embodiment, the top plate 130 may be formed from the same or a similar material as the flexible bag/sheets of material. In an embodiment, the top plate 130 is a polymeric rigid or semi-rigid plate or film patch. In an embodiment, the bag 100 may be manufactured with a variety of ports for insertion and positioning of various sensors and probes (not shown) within the flexible bag 100, and for connecting one or more fluid lines to the flexible bag 100 for fluids, gases, and the like, to be added or withdrawn from the flexible bag 100. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example. In order to function as a bioreactor, the bag may further comprise an impeller, suitably a magnetically driven impeller, comprising a plurality of magnets. The impeller can be rotatably attached to an impeller base plate and configured to be driven by an external magnetic drive. The bag can further be support by a rigid vessel, e.g. a cylindrical metal vessel, which can comprise a magnetic drive for driving the magnetic impeller.

The flexible bioprocessing bag 100 of the invention is advantageous in that it distributes stresses over four side welds instead of two tri-seam welds, which have heretofore been commonplace in the art. By welding a top panel 130 over the side welds in the area where the seams are folded over, additional pressure resistance can be obtained.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. As used herein to describe the present invention, directional terms such as “up”, down”, “upwards”, “downwards”, “upper”, “lower”, “top”, “bottom”, “vertical”, “horizontal”, “above”, “below” as well as any other directional terms, refer to those directions in the appended drawings.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A bioprocessing bag, comprising:
a plurality of panels joined to one another about respective edges of the plurality of sheets, by side welds; and
a top panel joined to upper edges of the plurality of panels by a convergence weld.

2. The bioprocessing bag of claim 1, wherein:
the plurality of panels is four panels.

3. The bioprocessing bag of claim 1, wherein:
the convergence weld is generally annular in shape.

4. The bioprocessing bag of claim 1, wherein:
the side welds define seams of the bioprocessing bag.

5. The bioprocessing bag of claim 1, wherein:
at least a portion of each of the seams is folded over; and
wherein the top panel overlaps the folded over seams.

6. The bioprocessing bag of claim 1, wherein:
the top panel is a polymeric rigid or semi-rigid plate.

7. The bioprocessing bag of any one of claim 1, wherein:
the top panel is a film patch.

8. The bioprocessing bag of claim 1, further comprising a plurality of ports.

9. The bioprocessing bag of claim 1, further comprising an impeller.

10. The bioprocessing bag of claim 9, wherein the impeller comprises a plurality of magnets and is rotatably attached to an impeller base plate and wherein the impeller is configured to be driven by an external magnetic drive.

11. A bioreactor comprising the bioprocessing bag of claim 1 mounted in a rigid support vessel.

12. The bioreactor of claim 11, comprising an external magnetic drive for driving an impeller in the bioprocessing bag.

13. A method of manufacturing the bioprocessing bag of one of claims 1-10, comprising the steps of:
i) cutting a plurality of panels to a generally rectangular shape with chamfered corners;
ii) joining the panels about their edges via side welds to define an interior space and forming a generally circular top opening;
iii) placing a generally circular bottom panel in a bottom opening and welding it to the lower edges of the panels using a convergence weld.
iv) placing a generally circular top panel in said top opening and welding it to the upper edges of the panels using a convergence weld.
